(19) United States
(12) Patent Application Publication
(10) Pub. No.: US 2022/0401346 A1
(43) Pub. Date: Dec. 22, 2022

(54) Hair removal admixture with wax, antiseptic, analgesic, moisturizing oil, and chemical depilatory: Hair removal chemical composite/creamy wax.

(71) Applicant: Mike Jenson Raj, Berrien Springs, MI (US)

(72) Inventor: Mike Jenson Raj, Berrien Springs, MI (US)

(21) Appl. No.: 17/304,300

(22) Filed: Jun. 17, 2021

(51) Int. Cl. A61K 8/92; A61Q 9/04; A61K 8/9794; A61K 8/22; A61K 8/46; A61K 31/196; A61K 31/616; A61K 8/9789; A61K 31/60; A61Q 17/00

(52) U.S. Cl. CPC A61K 8/922; A61Q 9/04; A61K 8/9794; A61K 8/22; A61K 8/463; A61K 8/46; A61K 31/196; A61K 31/616; A61K 8/9789; A61K 31/60; A61K 8/927; A61Q 17/005; A61K 2800/43; A61K 2800/882

(57) ABSTRACT

Hair removal Chemical composite/creamy wax. This waxing mixture is used to easily remove unwanted hair. To those seeking quick removal of hair without much pain, discomfort, sores, and the like. Helps to calm the skin during the hair removal procedure. Good for those seeking to do waxing by themselves, and doesn't need professional supervision. Quick hair removal waxing chemical composition, a waxing admixture for the 21st century. Less pain, more hair removal.

### BACKGROUND/FIELD OF INVENTION

Waxing is used to remove unwanted hair. This invention was done by the ancients as well. It is believed that waxing originated in Ancient Egypt, to less of an extent. This process was later refined by other ancient civilizations. Ancient Egyptians used waxing to remove excess hair. Their hot, and humid climate caused heat dissipation problems. The climate in those days, anciently, was quite different. Waxing has continued today, and is a multibillion-dollar industry. By 2019, it was reported that the worldwide waxing industry reached ˜$10 billion.

### SUMMARY OF INVENTION

Hair removal is a painful, cumbersome, and time-consuming work. Below is a list of claims about the admixture. This admixture is a chemical composition containing at least 5 different compounds, a 6th compound (colorant) is optional). 7th is also optional, it is for fragrance addition. This admixture will help to ease removal of hair. Once the compound is heated up, it needs to be liquid for spreading, then the waxing compound is applied to the cleaned area of the skin. The applied region of the skin is left to cool for 5-10 minutes max. The effect of the chemical composition needs time to act. The area under consideration will feel a very cooling sensation, followed by tingling sensation. The antiseptic alcohol provides the cooling effect, while the depilatory causes a burning sensation, followed by the analgesic compound, having a tingling or itching feeling. The sensations can be mild or very severe depending upon the percentage of the different components in the composition. For tough hard to remove hair, the composition will be different compared to those having silky smooth soft hair. This invention resolves many of the difficulties faced when using conventional wax.

### DETAILED DESCRIPTION/PROCEDURE OF FORMATION

This chemical admixture is a combination of more than 6 different chemical compounds, and so requires care and consideration when combining. Take soft or hard wax (those used for hair removal), and heat it until it becomes liquid.

Steps:
1. Heat wax until liquid. Make sure to weight the solid wax before heating. Also take into consideration the moles of it. To make it easy for admixture creation. The weight ratio is the key to mixing the admixture.
2. The heated liquid wax is continued to be heated until the addition of all the chemical compounds. Starting with moisturizing oil, coconut oil or aloe vera gel.
3. Then the antiseptic, like hydrogen peroxide or Sodium lauryl sulfate (SLS), is added.
4, After which the depilatory chemical is added, wax still being liquid. Like Calcium Thioglycolate or potassium thioglycolate.
5, Then the analgesic compound is added, Diclofenac or acetylsalicylic acid (ASA).
6. Add blue colorant or any desired color
7. Add fragrance (natural is preferred, like myrrh, etc.)
8. Add anti-inflammatory and anti-acne compound. Salicyclic acid at 2% of total weight of admixture.

The chemical composition of the creamy wax, is as follows. 95% to 98% wax (based on the removal agents, etc.), the other is the combinations as described above. In other instances the percentage of wax can be lowered to 80% or lower depending on the need of the wax being soft or hard. The temperature is kept bare minimum to melt the wax, at 90-100 F, and not more than what is needed. Excess heat can cause mixture not get homogenized. The wax combination is as follows: 60% carnauba wax and 40% beeswax.

Invention Overview (Quick Take-Away)

Purpose of Invention:

To remove hair.

What is claimed for admixture is:

1: Contains Wax (hard or soft, either will do). Mixture of carnauba wax and beeswax (60/40)

2: Contains moisturizing oil, powder, cream, or gel.

3: Contains antiseptic compound (not bactericide, but general antimicrobial)

4: Contains depilatory chemical (hair removal compound)

5: Contains analgesic compound (pain-killer)

6: Contains colorants

7: Contains fragrant

8: Contains anti-acne and anti-inflammatory compound
